(19) United States
(12) Patent Application Publication
(10) Pub. No.: US 2019/0331672 A1
(43) Pub. Date: Oct. 31, 2019

(54) HYDROGEL SENSOR DEVICES

(71) Applicant: Applied Biosensors, LLC, Salt Lake City, UT (US)

(72) Inventors: Nicholas Frazier, Salt Lake City, UT (US); Prashant Tathireddy, Salt Lake City, UT (US)

(21) Appl. No.: 16/176,813

(22) Filed: Oct. 31, 2018

Related U.S. Application Data
(60) Provisional application No. 62/579,741, filed on Oct. 31, 2017.

(51) Int. Cl. G01N 33/543; G01N 29/02; C08J 3/075

(52) U.S. Cl. CPC G01N 33/5436; G01N 29/022; C08J 2333/26; G01N 33/54373; C08J 2333/14; C08J 3/075

(57) ABSTRACT

A hydrogel sensor device can include a crosslinked hydrogel network having a first affinity ligand and a second affinity ligand positioned and configured to concurrently and reversibly bind to a common target molecule. The crosslinked hydrogel network can be further configured to decrease in volume with concurrent binding of the common target molecule. Generally, one or both of the first affinity ligand and the second affinity ligand are at least one of a protein, a peptide, and a synthetic biomimetic ligand. The hydrogel sensor device can also include a detector positioned to detect a change in volume of the crosslinked hydrogel network.

### RELATED APPLICATION(S)

The present application claims the benefit of U.S. Provisional Patent Application No. 62/579,741, filed on Oct. 31, 2017, which is incorporated herein by reference.

### GOVERNMENT INTEREST

This invention was made with government support under Grant No. IIP 1648079 awarded by the National Science Foundation. The government has certain rights in the invention.

### BACKGROUND

Biomanufacturing is a type of manufacturing that uses biological systems to produce commercially important biomaterials and biomolecules for use in medicines, food and beverage processing, industrial applications, etc. Bioproduction monitoring methods include off-line sampling from the bioreactor and analysis by instruments such as high performance liquid chromatography (HPLC), enzyme-linked immunosorbent assays (ELISA), radioimmune precipitation, surface plasmon resonance (SPR), and electrochemiluminescence (ECL), all of which are used to determine the concentration and viability of the biological products. In some cases, in-line optical sensors using frequency resonant energy transfer (FRET) have been developed to monitor pH and dissolved oxygen.

### SUMMARY

Hydrogel sensor devices can include a crosslinked hydrogel network having a first affinity ligand and a second affinity ligand positioned and configured to concurrently and reversibly bind to a common target molecule. The crosslinked hydrogel network can be further configured to decrease in volume with concurrent binding of the common target molecule. Generally, one or both of the first affinity ligand and the second affinity ligand are at least one of a protein, a protein fragment, a peptide, and a synthetic biomimetic ligand. The hydrogel sensor devices can also include a detector positioned to detect a change in volume of the crosslinked hydrogel network.

A method of detecting a target molecule can include concurrently and reversibly binding a common target molecule to a first affinity ligand and a second affinity ligand of a crosslinked hydrogel network to decrease a volume of the crosslinked hydrogel network. Generally, one or both of the first affinity ligand and the second affinity ligand are at least one of a protein, a peptide, and a synthetic biomimetic ligand or a synthetic chemical ligand with binding affinity to certain section of a target biological molecule. The method can also include detecting changes in the volume of the crosslinked hydrogel network.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying drawings and claims, or may be learned by the practice of the invention.

### BRIEF DESCRIPTION OF THE DRAWINGS

FIG. 1A illustrates a method of forming a crosslinked hydrogel network, in accordance with an example embodiment.

FIG. 1B illustrates the crosslinked hydrogel network of FIG. 1A with and without a target molecule reversibly bound thereto, in accordance with an example embodiment.

FIG. 2 illustrates an example of a target molecule bound to different affinity ligands, in accordance with an example embodiment.

FIG. 3 illustrates an example of a hydrogel sensor device, in accordance with an example embodiment.

FIG. 4A is a graph of surface plasmon resonance (SPR) signal after immobilization of affinity ligand samples before and after sterilization. \textcircled{1} and \textcircled{2} represent affinity ligand immobilization via biotin binding to neutravidin surface. The signal is unaffected by the sterilization procedures.

FIG. 4B is a graph of SPR signal of IgG binding to affinity ligand samples before and after sterilization. \textcircled{3} and \textcircled{4} represent IgG binding to immobilized affinity ligand samples. The signal is unaffected by the sterilization procedures.

FIG. 5 is a graph of hydrogel sensor response based on concentration of IgG. The concentration of IgG (lighter step curve) was cycled between 2.5 g/L and 0 g/L. The measured sensor response (darker curve) shows that the IgG sensor captures the cyclic changes in IgG concentration. Response time was not optimized for these tests. A few minor experimental artifacts are seen as sharp spikes in the sensor data.

FIG. 6A is a graph of the time-dependent hydrogel sensor response (darker curve) to stepwise increases in IgG concentration (lighter step curve) between 0.1 g/L and 10 g/L.

FIG. 6B is a graph of the time-dependent hydrogel sensor response (darker curve) to stepwise decreases in IgG concentration (lighter step curve) between 0.1 g/L and 10 g/L.

FIG. 7 is a graph of IgG concentrations measured using an in-situ continuous hydrogel-based magnetic sensor as compared with offline testing results obtained using industry standards such as CEDEX and Nanodrop analytical equipment.

FIG. 8 is a graph of the time-dependent response of the hydrogel sensor to stepwise changes in IgG concentration. Since the media had previously been used for cell culture, it likely contained host cell proteins.

These drawings are provided to illustrate various aspects of the invention and are not intended to be limiting of the scope in terms of dimensions, materials, configurations, arrangements or proportions unless otherwise limited by the claims.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “an affinity ligand” includes reference to one or more of such materials and reference to “detecting” refers to one or more such steps.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, “affinity ligand” refers to a chemical moiety which exhibit a reversible binding to at least one section of a target molecule.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, and combinations of each.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Hydrogel Sensor Devices

As described above, biomanufacturing is a type of manufacturing that uses biological systems to produce commercially important biomaterials and biomolecules for use in medicines, food and beverage processing, industrial applications, etc. Biomanufacturing of monoclonal antibodies (mAbs), more specifically immunoglobulin G (IgG), encompasses a large portion of the biopharmaceutical market. Bioproduction monitoring methods typically include off-line sampling from the bioreactor and analysis by instruments such as high performance liquid chromatography (HPLC), enzyme-linked immunosorbent assays (ELISA), radioimmune precipitation, surface plasmon resonance (SPR), and electrochemiluminescence (ECL), any of which can be used to determine the concentration and quality of the biomolecules produced. However, these methods are slow, can add downtime costs to run the instrument and analyze results, and can require expensive equipment to run and maintain. Sampling can also disrupt the biomanufacturing process, possibly leading to contamination and subsequently total loss of the batch. Thus, there is a need for in-line sensors, which can be disposable, that are capable of accurately measuring the concentration of biomolecules, such as mAbs, produced within a commercial bioreactor in real-time.

An in-line stimuli-responsive hydrogel sensor is disclosed herein that can improve process efficiency and reduce the cost of manufacturing. More specifically, the stimuli-responsive hydrogel sensor can perform the monitoring function in a bioreactor by using affinity ligands that specifically bind to a target molecule, such as mAbs, or other suitable biomolecules. Varying concentrations of target molecules can cause varying degrees of shrinking or swelling of the hydrogel network, which can be monitored by methods including optical sensing, mechanical or pressure sensing, acoustic (ultra-sound) testing, electrical sensing (change in electrical properties in hydrogel due to volume change, impedance, conductivity, dielectric properties, resonant electro-magnetic frequencies), sensing by the means of micro-fluidic systems (change in flow in microfluidic channel or a chromatographic column due to change in hydrogel volume), magnetic sensing the like, or a combination thereof. The hydrogel sensor devices described herein can be used with a variety of bioreactors, including single-use polymeric vessels, conventional non-disposable bioreactors, other suitable bioreactors, clinical, fermentation, food and beverage processing, water quality, point of care, monitor presence of target molecules, and the like, to monitor production of a target molecule.

In further detail, a hydrogel sensor device can include a crosslinked hydrogel network and a detector positioned to detect a change in volume of the crosslinked hydrogel network. The crosslinked hydrogel network can include a first affinity ligand and a second affinity ligand positioned and configured to concurrently and reversibly bind to a common target molecule. The crosslinked hydrogel network can be further configured to change in volume with concurrent binding of the common target molecule. Generally, one or both of the first affinity ligand and the second affinity ligand can be at least one of a protein, a protein fragment, a peptide a synthetic biomimetic ligand, and or a synthetic chemical ligand with binding affinity to certain section of a target biological molecule.

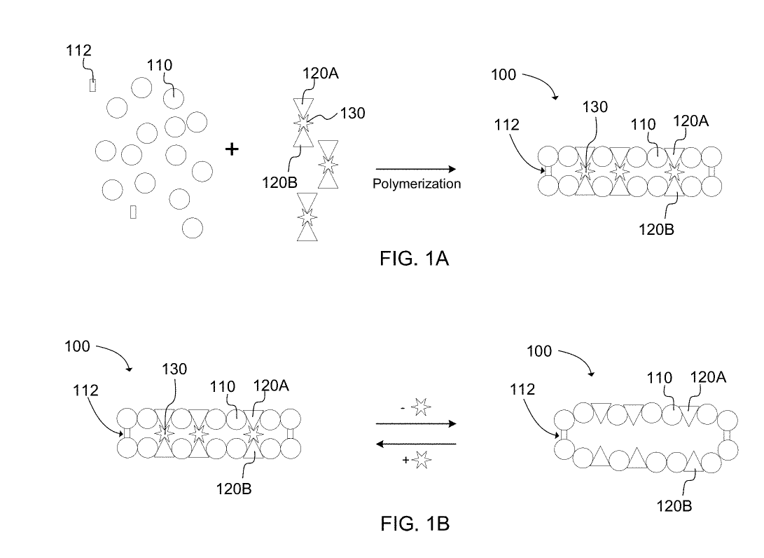

The crosslinked hydrogel network can be formed in a variety of ways. One non-limiting example is illustrated in FIG. 1A. A plurality of primary monomers or repeat units 110 can be co-polymerized with first affinity ligand 120A and second affinity ligand 120B. It is noted that by first crosslinking the affinity ligand pairs 120A, 120B with the target molecule 130 to form a crosslinked precursor structure, the crosslinked precursor structure can be co-polymerized with the monomers 110 to suitably position affinity ligands 120A, 120B relative to one another within the crosslinked hydrogel network 100 to be able to concurrently bind to the target molecule 130. However, other methods of suitably positioning affinity ligand pairs 120A, 120B can also be employed. Further, individual polymer chains can be covalently crosslinked together using a crosslinking agent or crosslinking monomer 112. A number of affinity ligands, 120A and 120B in a unit volume of hydrogel determines the concentration range that the hydrogel will respond to in an application. Therefore the sensitivity range of the hydrogel can be tailored, within reason (polymerization limitations), by the concentration of affinity ligands in the hydrogel.

Thus, as illustrated in FIG. 1B, where target molecule 130 is released from the crosslinked hydrogel network 100, the crosslinked hydrogel network 100 can swell or increase in volume. However, when the first affinity ligand 120A and second affinity ligand 120B concurrently bind target molecule 130, the crosslinked hydrogel network can shrink or decrease in volume. It is noted that crosslinking agent or monomer 112 can limit the degree of swelling and preserve the hydrogel 100 in an insoluble state. As such, the crosslinked hydrogel network 100 can include both reversible and permanent crosslinks. Although this illustration shows a decrease in volume as a response to binding with the target molecule, the reverse can also occur. For example, in some cases, the crosslinked hydrogel network can increase in volume upon binding with the target molecule.

Thus, as described above, the crosslinked hydrogel network responds to changes in concentration of the target molecule through forming and breaking of reversible crosslinks that leads to shrinking or swelling of the hydrogel. The reversible crosslinks can lead to shrinking and swelling of the hydrogel based on changes in local concentration of the target molecule. In the presence of the target molecule, affinity ligand pairs can bind to different regions of the target molecule to create a reversible crosslink between two polymer chains. The formation of crosslinks brings the polymer chains closer together and the hydrogel network shrinks. As the concentration of the target molecule increases, so does the number of reversible crosslinks causing the gel to shrink further. The amount of shrinking can be correlated to a local concentration of the target molecule. Where the concentration of the target molecule decreases, the equilibrium shifts to a lower number of reversible crosslinks and the hydrogel swells. Based on the concentration, an equilibrium of binding and dissociation occurs, which can lead to a certain degree of swelling or shrinking. Degree of volume change can vary depending on the specific polymer, affinity ligands, and concentration of target molecule. However, as a general guideline volume changes up to 2 volume %, in other cases up to about 10 volume % and in some cases up to 50 volume % from an initial hydrated state can be obtained.

It is noted that the crosslinked hydrogel network can be obtained by various modifications and polymerization methods. In some specific examples, the crosslinked hydrogel network can be obtained by co-polymerization of a primary monomer (e.g. acrylamide monomer, for example), a crosslinking monomer (e.g. methylenebisacrylamide (MBAA) monomer, for example), first affinity ligand monomer, and second affinity ligand monomer. These four monomers can be co-polymerized using UV initiators, thermal initiators, or the like to form the crosslinked hydrogel network. The amount and ratios of the various monomers and the initiator can be varied to produce a gel with optimal sensitivity and response magnitude. The various monomers of the crosslinked hydrogel network can be positioned randomly with respect to one another, in blocks with respect to one another, or in any other suitable configuration with respect to one another.

In further detail, a variety of suitable primary backbone monomers or repeat units can be employed in preparing the crosslinked hydrogel network. Generally, any primary monomers or repeat units that can be co-polymerized with the affinity ligands disclosed herein and that are suitable for preparing a crosslinked hydrogel network that swells and shrinks in response to concurrent binding of affinity ligand pairs can be used. Some non-limiting examples of primary monomers can include 2-hydroxyethyl methacrylate (HEMA), 2-hydroxypropyl methacrylate (HPMA), acrylamide (AAm), acrylic acid (AAc), N-isopropylacrylamide (NIPAm), polyethylene glycol monoacrylate (PEGMA), methoxyl polyethylene glycol monoacrylate (mPEGMA), the like, or a combination thereof. In some specific examples, the primary monomer or repeat unit can include AAm, an N-substituted AAm, or a combination thereof. In some examples, the hydrogel can be formed of a hydrophilic polymer.

The primary monomer or repeat unit can generally be present in the crosslinked hydrogel network in an amount greater than 70 mol %, or greater than 80 mol %. In some specific examples, the primary monomer or repeat unit can be present in the crosslinked hydrogel network in an amount of from about 89 mol % to about 99.99 mol %, and in some cases 80 mol % to 95 mol %, and in other cases from 85 mol % to about 94 mol %.

A variety of suitable crosslinking agents or crosslinking monomers can also be incorporated into the crosslinked hydrogel network. Generally, any suitable crosslinking agent or crosslinking monomer that is suitable to covalently crosslink individual polymer chains of the hydrogel networks described herein can be employed. Non-limiting examples can include NN′-methylenebis(acrylamide) (MBAA), N,N-diallylacrylamide, ethylene glycol diacrylate (EGDA), polyethylene glycol diacrylate (PEGDA), polyethylene glycol dimethacrylate (PEGDMA), and combinations thereof. In some specific examples, the crosslinking monomer can be or include MBAA.

The crosslinking monomer can be present in the crosslinked hydrogel network in a sufficient amount to maximize the detectable volume change of the hydrogel, but without loss of structural integrity that can lead to polymer chain segregation. In some specific examples, the crosslinking monomer can be present in the crosslinked polymer network in an amount of from about 0.01 mol % to about 10 mol %, and in some cases from 0.25 mol % up to about 5 mol %.

A variety of suitable affinity ligands can also be incorporated or co-polymerized into the crosslinked hydrogel network. Specifically, affinity ligand pairs including a first affinity ligand and a second affinity ligand can be positioned within or as part of the crosslinked hydrogel network to concurrently bind to a target molecule. It is noted that the two affinity ligands can be modified as a functional group on a monomer that can be incorporated or copolymerized into the crosslinked hydrogel network. For example, each affinity ligand can be added to an acrylic acid monomer, an acrylate acrylate monomer, an acrylamide monomer, or other suitable monomer (e.g. those listed as suitable primary monomers) while substantially preserving individual affinity ligands' binding affinities.

In some examples, a first affinity ligand and the second affinity ligand can be the same. In some other examples, a first affinity ligand and the second affinity ligand can be different. Where the first affinity ligand and the second affinity ligand are different, in some examples, the first affinity ligand and the second affinity ligand can be configured to generally, or in some cases universally, bind to the target molecule at one or more of a plurality of binding sites on the target molecule. In some other examples, the first affinity ligand and the second affinity ligand can be configured to bind to different parts of the target molecule. For example, in some cases, the first affinity ligand can be configured to bind to a first portion of the target molecule and the second affinity ligand can be configured to bind to a second portion of the target molecule.

As described above, typically one or both of the first affinity ligand and the second affinity ligand can be at least one of a protein, a protein fragment, a peptide, a synthetic biomimetic ligand, and or a synthetic chemical ligand with binding affinity to certain section of a target biological molecule. In some specific examples, the first affinity ligand, the second affinity ligand, or both can be configured to bind to the crystallizable fragment (Fc) region of IgG or another antibody. In some additional specific examples, the first affinity ligand, the second affinity ligand, or both can be configured to bind to the antigen-binding fragment (Fab) region of IgG or another antibody. Non-limiting examples of suitable affinity ligands can include protein A, protein G, protein AG, protein Z, protein LG, protein LA, protein L, peptide PAM, peptide TWKTSRISIF, petide FGRLVSSIRY, peptide EPIHRSTLTALL, peptide APAR, peptide HWRGWV, peptide HYFKFD, peptide HFRRHL, peptide HWCitGWV, peptide D₂AAG, peptide DAAG, peptide cyclo[Nα-Ac)S(A)-RWHYFK-Lact-E], peptide cyclo[(Nα-Ac)-Dap(A)-RWHYFK-Lact-E], peptide cyclo[Link-M-WFRHYK], peptide FcBP-1, peptide FcBP-2, peptide FcRM, peptide Fc-III, peptide Fc-III-4C, ligand 8/7, the like, or a combination thereof. In some specific examples, the first affinity ligand or the second affinity ligand can include a Fab-binding affinity ligand, such as protein A, protein G, protein L, ligand 8/7, the like or a combination thereof. In some additional specific examples, the first affinity ligand or the second affinity ligand can include an Fc-binding affinity ligand, such as protein A, protein G, protein AG, protein Z, protein LG, protein LA, peptide PAM, peptide FcBP-1, peptide FcBP-2, peptide FcRM, peptide Fc-III, peptide Fc-III-4C, ligand 88/2, the like, or a combination thereof These and additional affinity ligands are described in Choe, W. et al., Fc-Binding Ligands of Immunoglobulin G: An Overview of High Affinity Proteins and Peptides. Materials 2016, 9, 994; and Roque A. C. A. et al., An artificial protein L for the purification of immunoglobulins and Fab fragments by affinity chromatography. Journal of Chromatography A, 2005, 1064, 157, each of which is incorporated herein by reference. In some cases, the affinity ligand can include an aptamer (e.g. oligonucleotides or peptides). Additionally, other proteins can be developed to bind to specific regions of the IgG molecule typically used in enzyme-linked immunosorbent assay (ELISA). These include antibodies and antibody fragments. The Fab region can be separated from the antibody and used as an affinity ligand or aptamer. For example, anti-human IgG (including but not limited to peroxidase antibody, Fc specific antibody, Fab specific antibody, alkaline phosphatase antibody, FITC antibody, F(ab′)2 fragment-R-Phycoerythrin antibody, biotin antibody, Cy3 antibody, gamma-chain specific antibody, agarose antibody, fluorescein antibody, rhodamine antibody, peroxidase antibody, conjugates thereof, and the like can be used. Specific commercial examples such as A0170, I2136, I8885, I5260, I9010, A9544, A0293, F9512, A6029, A8419, A8667, A8792, F5512, F3512, P8047, B3773, C2571, A1543, A8542, I1886, I1011, B1140, I9135, SAB3701275, I3382, I9881, I1136, I3391, F1641, P9170, F4512, A2064, I6760, I6260, SAB4200682, A3316, A3187, A3150, I3266, calreticulin, (all available from Millipore Sigma) can be used. Further, the binding regions of protein A, protein G, and protein L can also be separated and used instead of the whole protein. Lastly, developmental technologies such as systematic evolution of ligands by exponential enrichment (SELEX) continue to produce new aptamers for binding to IgG. Such ligands can also be readily incorporated and used as suitable affinity ligands.

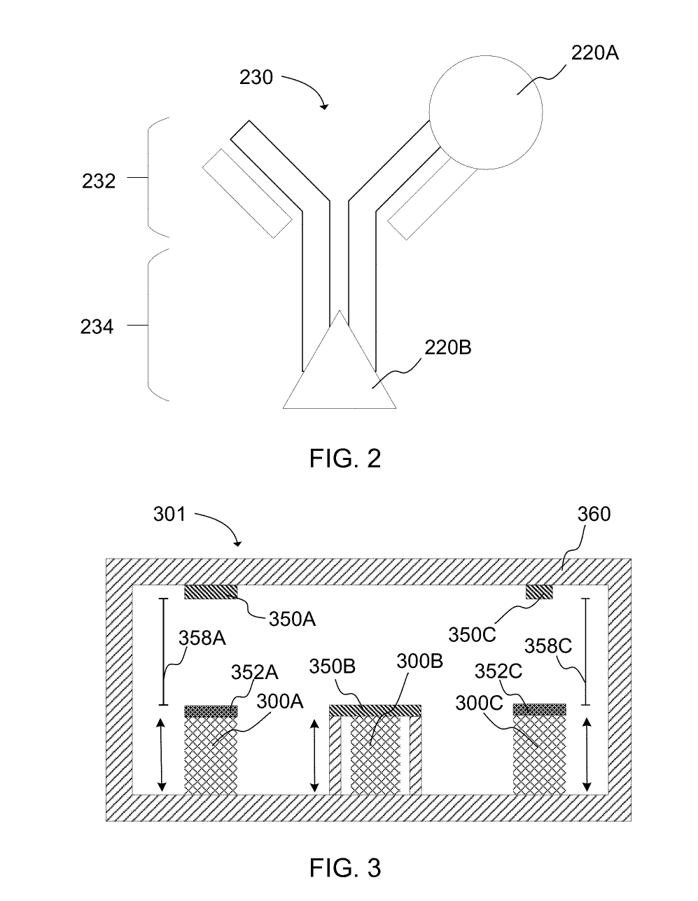

One non-limiting example of an affinity ligand pair binding to a target molecule is illustrated in FIG. 2. Specifically, as illustrated in FIG. 2, target molecule 230 is an antibody. The antibody has an antigen-binding fragment (Fab) region 232 and a crystallizable fragment (Fc) region 234. The first affinity ligand 220A can be configured to specifically bind to the antibody at the Fab region 232 and the second affinity ligand can be configured to specifically bind to the antibody at the Fc region 234. However, in some cases, the first affinity ligand 220A and the second affinity ligand 220B can both be configured to bind to the Fab region and the Fc region of the antibody. Again, this is one non-limiting example that can be applied to target molecules in addition to antibodies per se. Various biomolecules can have a plurality of binding sites to which affinity ligand pairs of a crosslinked hydrogel network can be configured to concurrently bind to reversibly decrease a volume thereof. For example, the target molecule can be or include a virus, a protein, a nucleic acid, an amino acid, an enzyme, an antibody, small molecule (e.g. glucose), the like, or a combination thereof. In some specific examples, the target molecule can be or include an antibody. In some further examples, the target molecule can be or include IgG.

Generally, the first affinity ligand and the second affinity ligand can be respectively present in the crosslinked hydrogel network in an amount less than 5 mol %. In some specific examples, the first affinity ligand and the second affinity ligand can be respectively present in the crosslinked hydrogel network in an amount of from about 0.001 mol % to about 5 mol % and in some cases up to 1 mol %.

It is noted that the first affinity ligand and the second affinity ligand can have an affinity for the target molecule that is sufficiently high to generate crosslinks that are strong enough to change a volume of the hydrogel network where sufficient crosslinks are present. However, the affinity of the first affinity ligand and the second affinity ligand for the target molecule can also be sufficiently low that the crosslinks can be reversible. For example, as a general guideline, suitable dissociation constants (K_{d}) for these aptamers can range from 500 μM to 0.1 nM.

A detector can be positioned to detect changes in volume of the crosslinked hydrogel network based on formation and dissociation of reversible crosslinks with the target molecule. A variety of suitable detectors can be used. Non-limiting examples of suitable detectors can include optical detectors, pressure detectors, magnetometers, the like, or a combination thereof.

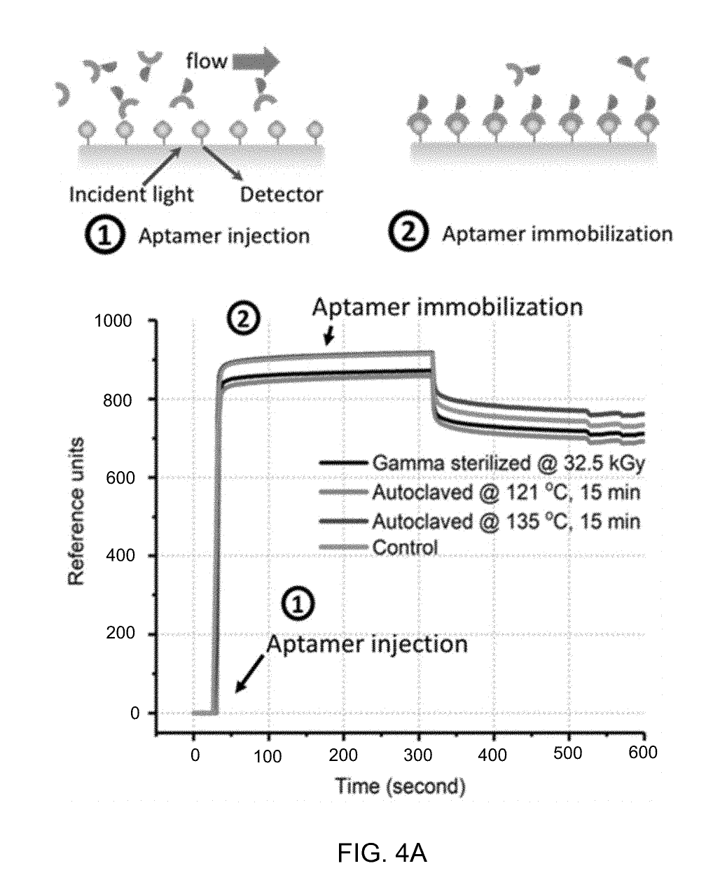

For example, FIG. 3 illustrates a hydrogel sensor device 301 including a housing 360 and a plurality of crosslinked hydrogel networks 300A, 300B, 300C respectively associated with individual detectors 350A, 350B, 350C. As an inline sensor, an inlet 370 can be oriented to accept incoming fluids to allow fluids to fluidly contact individual hydrogel networks. Fluids can then exit the housing via outlet 372. In further detail, magnetometer 350A can be positioned to detect a volume change in crosslinked hydrogel network 300A. More specifically, crosslinked hydrogel network 300A can be coupled to a metal sheet 352A. Changes in volume of crosslinked hydrogel network 300A, based on a local concentration of the target molecule, can change a distance 358A between the metal sheet 352A and the magnetometer 350A. These changes in distance 358A between the metal sheet 352A and the magnetometer 350A can be detected by magnetometer 350A and correlated to a local concentration of the target molecule. Additional examples and details of magnetometers can be found in U.S. patent application Ser. No. 15/006,172, filed Jan. 26, 2016, which is incorporated herein by reference.

Similarly, pressure sensor 350B is positioned to detect changes in volume of crosslinked hydrogel network 300B. For example, detector 350B can represent a strain gauge coupled to the crosslinked hydrogel network 300B such that a change in volume of the crosslinked hydrogel network 300B can impose a detectable stress on pressure detector 350B. The degree of stress detected by the pressure detector 350B can be correlated to a local concentration of the target molecule. Non-limiting examples of pressure sensors can include piezoresistive strain gauges, capacitive pressure detectors, electromagnetic pressure sensors, piezoelectric pressure sensors, strain gauges, potentiometric pressure detectors, the like, or a combination thereof.

Additionally, optical detector 350C can be positioned to detect changes in volume of crosslinked hydrogel network 300C. In some examples, optical detector 350C can represent an interferometric detector or other suitable optical distance detector and 352C can represent a mirror or other suitable surface to reflect a coherent beam of light back to the optical detector 350C to measure a change in distance 358C between the mirror 352C and the optical detector 350C. Other suitable optical distance measurements can also be employed. Alternatively, object 352C can represent a transparent surface or can be absent from the device 301. Where this is the case, optical detector 350C can be configured to detect a luminescent response (e.g. fluorescence, chemiluminescence, etc.) based on binding of the target molecule to the crosslinked hydrogel network.

In some examples, the hydrogel sensor device can include more than one detector type, at least one of which is configured to measure a change in volume of the crosslinked hydrogel network. In some examples, the hydrogel sensor device can include a detector that does not directly measure a change in volume (e.g. detection of luminescent response, for example), but that can corroborate data points collected by another detector configured to measure a change in volume. In other examples, the hydrogel sensor device can include a single detector type.

Corresponding detectors can produce data in the form of a detector response signal (e.g. voltage change, fluorescence signal, intensity, magnetic field value, etc) which can be collected by a memory device. The memory device can store such data for later analysis or simultaneously communicate collected data to a computing device. For example, a computing device on which collection, correlation, analysis, and/or output of this technology can be used. In one example, collected data can be compared to a reference dataset. The reference dataset can be produced through subjecting the detectors to known concentrations of target molecules to obtain data of detector response as a function of concentration. In this manner, collected data can be compared with the reference dataset to determine a corresponding target molecule concentration in an unknown fluid. In another example, the reference data with known concentrations can be used to train a machine-learning algorithm and later the algorithm can be used to predict true concentrations based on the hydrogel volume-change signal. The machine-learning algorithm can be based on wide range of models from simple linear-model techniques such as least-square estimates to advanced models such as artificial neural-networks.

The computing device may include one or more processors that are in communication with memory devices. The computing device may include a local communication interface for the components in the computing device. For example, the local communication interface may be a local data bus and/or any related address or control busses as may be desired.

The memory device may contain modules that are executable by the processor(s) and data for the modules. The modules may execute functions that perform the methods described herein. A data store may also be located in the memory device for storing data related to the modules and other applications along with an operating system that is executable by the processor(s).

The computing device may also have access to I/O (input/output) devices that are usable by the computing devices. An example of an I/O device is a display screen that is available to display output from the computing device. Networking devices and similar communication devices may be included in the computing device. The networking devices may be wired or wireless networking devices that connect to the internet, a LAN, WAN, or other computing network.

The components or modules that are shown as being stored in the memory device may be executed by the processor(s). The term “executable” may mean a program file that is in a form that may be executed by a processor. For example, a program in a higher level language may be compiled into machine code in a format that may be loaded into a random access portion of the memory device and executed by the processor, or source code may be loaded by another executable program and interpreted to generate instructions in a random access portion of the memory to be executed by a processor. The executable program may be stored in any portion or component of the memory device. For example, the memory device may be random access memory (RAM), read only memory (ROM), flash memory, a solid state drive, memory card, a hard drive, optical disk, floppy disk, magnetic tape, or any other memory components.

The processor may represent multiple processors and the memory device may represent multiple memory units that operate in parallel to the processing circuits. This may provide parallel processing channels for the processes and data in the system. The local interface may be used as a network to facilitate communication between any of the multiple processors and multiple memories. The local interface may use additional systems designed for coordinating communication such as load balancing, bulk data transfer and similar systems.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, non-transitory media such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. By way of example and not limitation, communication media includes wired media such as a wired network or direct-wired connection and wireless media such as acoustic, radio frequency, infrared and other wireless media. The term computer readable media as used herein includes communication media.

The present disclosure also describes a method of detecting a target molecule. The method can include concurrently and reversibly binding a common target molecule to a first affinity ligand and a second affinity ligand of a crosslinked hydrogel network to decrease a volume of the crosslinked hydrogel network. One or both of the first affinity ligand and the second affinity ligand are at least one of a protein, a protein fragment, a peptide, a synthetic biomimetic ligand, the like, or a combination thereof. The method further includes detecting changes in volume of the crosslinked hydrogel network.

It is noted that when discussing the hydrogel sensor devices and the methods of detecting a target molecule, each of these discussions can be considered applicable to one another whether or not they are explicitly discussed in the context of that example. Thus, for example, when discussing a detector related to hydrogel sensor devices, such disclosure is also relevant to and directly supported in the context of methods of detecting a target molecule, and vice versa.

As described above, the crosslinked hydrogel network can change in volume in response to a local concentration of a target molecule. As such, the hydrogel sensor devices described herein can be incorporated into a variety of bioreactors, or other suitable devices where it is desirable to monitor a concentration of a target molecule. Thus, a target molecule can be contacted with the crosslinked hydrogel network or the crosslinked hydrogel network can be exposed to the target molecule to induce detectable changes in volume of the crosslinked hydrogel network. These changes in volume can be correlated to an amount or local concentration of the common target molecule.

Depending on the particular application of the hydrogel sensor device, data points can be collected at a variety of time points to determine total local concentrations of the target molecule at a given time point, rate of change of the local concentration, etc. The specific periodicity or frequency of the data points can be optimized based on a particular application.

In some specific examples, local concentration data (e.g. total concentration, rate of concentration change, etc.) can be transmitted to a remote device, such as a computer, a server (e.g. local data server, cloud server, etc.), a smart device, or the like for evaluation and monitoring by an end user or for data storage. In some examples, the hydrogel sensor device can be physically connected to the remote device using wires or the like. In other examples, the hydrogel sensor device can be wirelessly connected to the remote device.

Where this is the case, a variety of suitable wireless protocols can be employed to transmit the local concentration data to the remote device. Non-limiting examples can include Bluetooth® (e.g. Bluetooth® low energy (LE)), Zigbee, other suitable IEEE protocols, WiFi, WiMAX®, the like, or other suitable wireless protocols.

In some further examples, the local concentration data can be processed by the remote device and the remote device can transmit instructions to a controller module of the bioreactor or other suitable device to alter and/or maintain one or more parameters, such as temperature, mixing rate, pH, osmolarity, etc. In some specific examples, the remote device can include a controller module of the bioreactor. Thus, in some examples, the local concentration data can be used to generate an automatic feedback response.

### EXAMPLES

### Example 1—Effect of Sterilization Procedures on Aptamer Binding Affinity

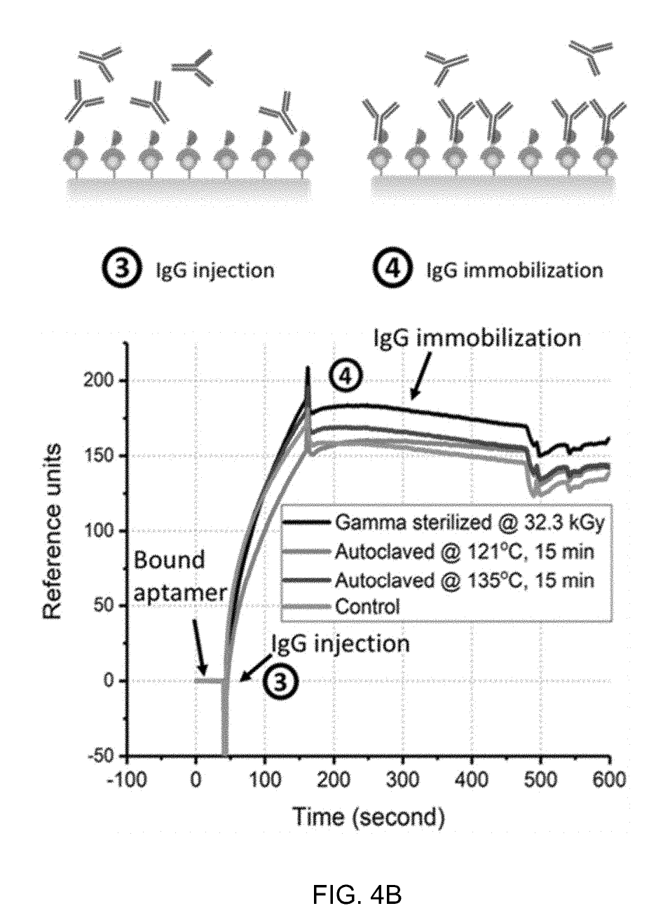

Surface plasmon resonance (SPR) technique was used to determine IgG-binding affinity of selected aptamers before and after sterilization. Aptamers were immobilized to a neutravidin surface using biotin binding. IgG samples were then bound to immobilized aptamer samples. FIG. 4A presents a graph of the SPR signal after immobilization of aptamers. FIG. 4B presents a graph of the SPR signal of IgG binding to immobilized aptamers. As shown in FIGS. 4A-4B, the signal is unaffected by the sterilization procedures. SPR is a technique used for determining binding of an analyte (i.e., IgG) to a ligand (i.e., ap-tamer). Both aptamers were modified with a biotin functional group and immobilized onto a gold surface modified with neutravidin (biotin capture surface) as depicted in FIG. 4A. During SPR immobilization of the aptamers, the molecules bind to the surface of the gold, changing the surface plasmon resonance of the gold layer by altering the reflected angle of laser light that shines through a prism onto the gold surface. These changes are detected and used to determine whether the aptamer has bound to the surface (FIG. 4A). Once the aptamer is immobilized, the surface is washed to remove unbound peptides, which is reflected in a slight decrease in signal in FIG. 4A after 300 sec. Finally, free IgG is flowed over the immobilized aptamer, and binding is detected in the signal (FIG. 4B). The aptamers were autoclaved at 121° C. and 135° C. for 15 minutes, and binding to IgG was measured by SPR. Additionally, a different set of aptamers were treated with 32.3 kGy of gamma irradiation. The degree of immobilization of sterilized aptamers (100 nM concentration) is shown in FIG. 4A. Each treatment group has similar signal intensities, indicating that comparable amounts of ap-tamer are immobilized. Next, the ability of sterilized aptamers to bind IgG was tested (FIG. 4B). No significant differences in binding were observed for the different groups after sterilization.

### Example 2—Evaluation of Example Hydrogel Sensor Device

A crosslinked hydrogel network was prepared including acylamide primary monomers, N,N′-methylenebis(acrylamide) crosslinking monomers, ligand 8/7 as aptamer for the Fab region of IgG, and peptide Fc-III-4C as aptamer for the Fc region of IgG. A magnetometer detector was employed to detect changes in volume of the crosslinked hydrogel network.

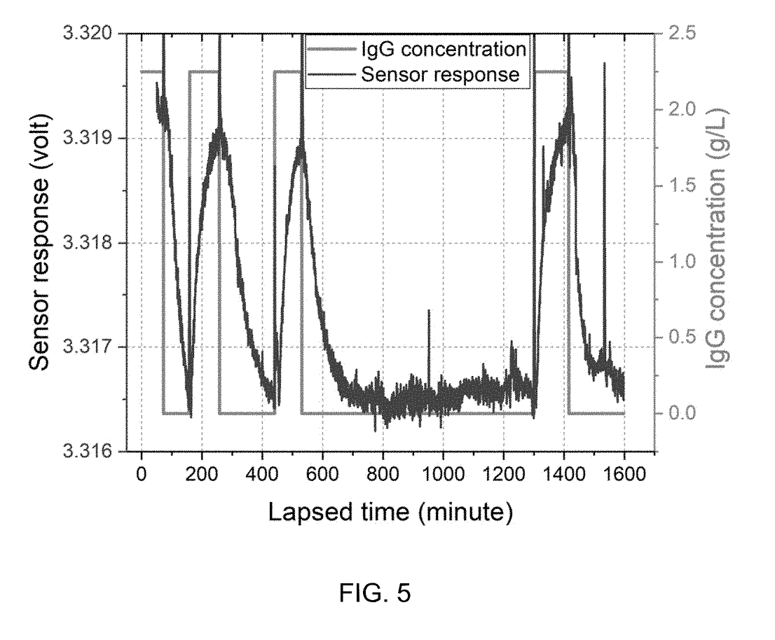

As presented in FIG. 5, IgG was able to reversibly bind to the crosslinked hydrogel network. More specifically, the concentration of IgG (lighter step curve) was cycled between 2.5 g/L and 0 g/L. The measured sensor response (darker curve) shows that the hydrogel sensor device captures the cyclic changes in IgG concentration. The response time was not optimized for these tests. A few minor experimental artifacts are seen as sharp spikes in the sensor data.

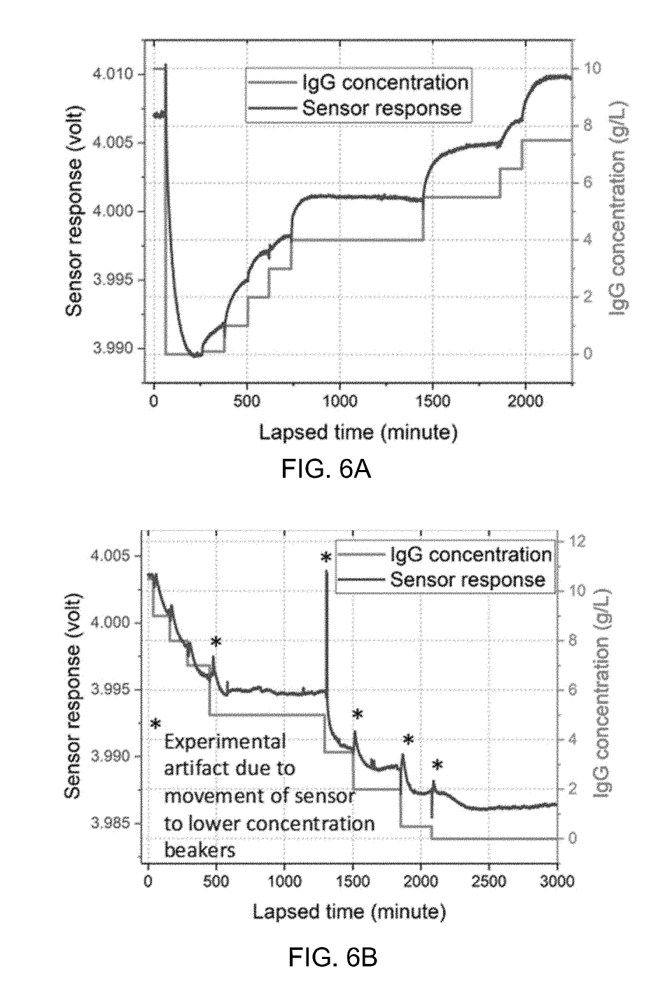

Further, as presented in FIGS. 6A-6B, the hydrogel sensor device can detect IgG over a commercially-relevant concentration range. More specifically, FIG. 6A presents a time-dependent response of the sensor (darker curve) to stepwise increases in IgG concentration (lighter step curve) between 0.1 g/L and 10 g/L and FIG. 6B presents a time-dependent response of the sensor (darker curve) to stepwise decreases in IgG concentration (lighter step curve) between 0.1 g/L and 10 g/L.

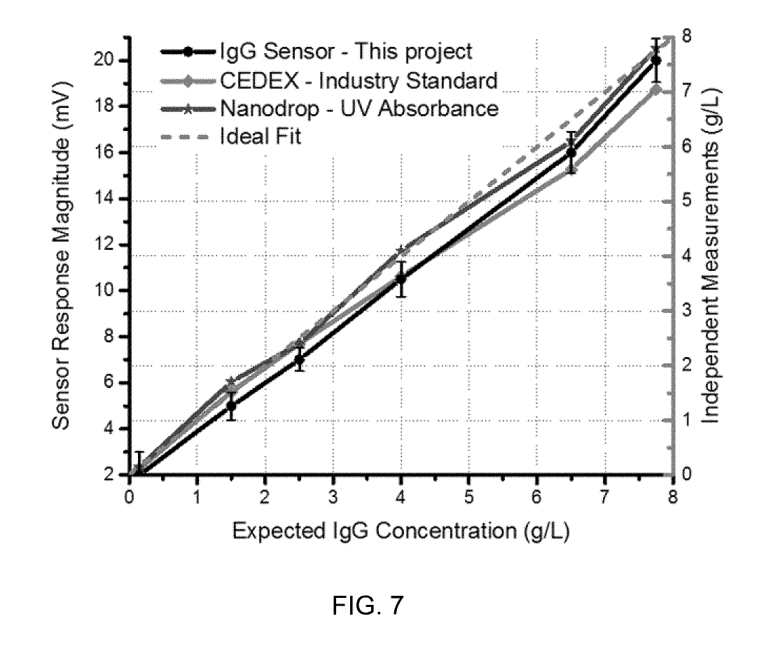

To further validate the hydrogel sensor device, sensor data was compared to offline “Gold Standard” measurements of IgG: In further detail, IgG concentrations were measured using the in-situ continuous hydrogel-based magnetic sensor. These values were compared with offline testing results obtained using industry standards such as CEDEX and Nanodrop analytical equipment. Results are presented in FIG. 7.

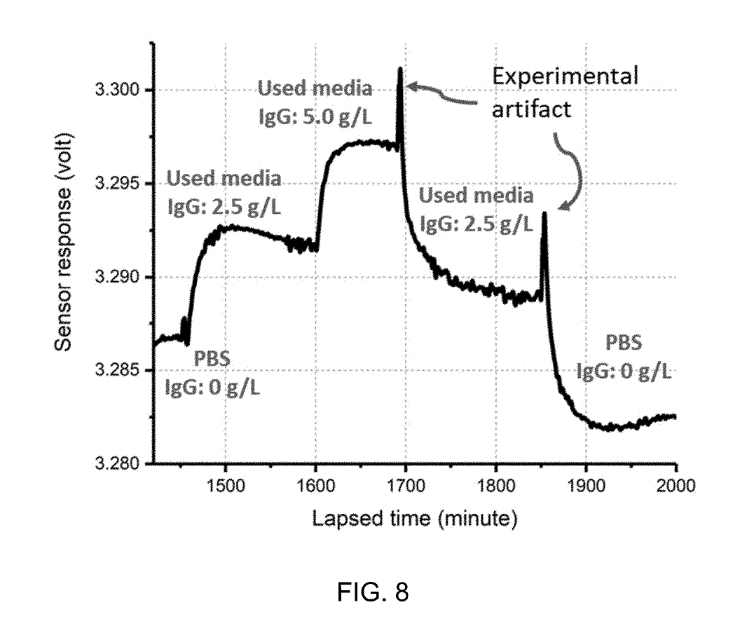

To further evaluate the hydrogel sensor device, the device was placed in a commercial cell growth medium to determine whether the sensor could measure IgG concentrations in a commercial environment. FIG. 8 demonstrates that the hydrogel sensor device can measure the IgG concentration in a commercial cell growth medium. More specifically, the time-dependent response of the hydrogel sensor device to stepwise changes in IgG concentration is presented in FIG. 8. It is noted that, since the media had previously been used for cell culture, it likely contained host cell proteins.

The hydrogel-based sensor was tested in cell culture medium that had previously been used in a commercial 14-day cell culture run. At the end of this run, 10 L of unfiltered media containing approximately 2.5 g/L IgG with lysed cells. The sensor was first stabilized in PBS and then exposed to the used cell culture media. After a brief stabilization period in the cell culture media, IgG was added to increase the concentration of the media to 5 g/L, and the hydrogel response was observed (FIG. 8). The increase in the signal in FIG. 8 demonstrates the IgG-sensitive hydrogel ability to respond in a commercial bioreactor environment. After the sensor signal stabilized, the sensor was returned to the original (used) cell culture media with 2.5 g/L of IgG. The observed decrease in the sensor signal further demonstrates the sensor's reversibility in a commercial cell culture environment. Lastly, the sensor was returned to PBS, in which the same signal reversal was observed.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A hydrogel sensor device, comprising:
a crosslinked hydrogel network including a first affinity ligand and a second affinity ligand positioned and configured to concurrently and reversibly bind to a common target molecule, wherein the crosslinked hydrogel network is further configured to decrease in volume with concurrent binding of the common target molecule, and wherein one or both of the first affinity ligand and the second affinity ligand are at least one of a protein, a protein fragment, a peptide, a synthetic biomimetic ligand, and a synthetic chemical ligand; and
a detector positioned to detect a change in volume of the crosslinked hydrogel network.

2. The hydrogel sensor device of claim 1, wherein the crosslinked hydrogel network further comprises a primary backbone monomer and a crosslinking monomer.

3. The hydrogel sensor device of claim 2, wherein the primary backbone monomer is selected from the group consisting of: 2-hydroxyethyl methacrylate (HEMA), 2-hydroxypropyl methacrylate (HPMA), acrylamide (AAm), acrylic acid (AAc), N-isopropylacrylamide (NIPAm), polyethylene glycol monoacrylate (PEGMA), methoxyl polyethylene glycol monoacrylate (mPEGMA), and combinations thereof.

4. The hydrogel sensor device of claim 2, wherein the primary backbone monomer is present in the crosslinked hydrogel network in an amount of from about 89 mol % to about 99.99 mol %.

5. The hydrogel sensor device of claim 2, wherein the crosslinking monomer is selected from the group consisting of: N,N′-methylenebis(acrylamide) (MBAA), NN-diallylacrylamide, ethylene glycol diacrylate (EGDA), polyethylene glycol diacrylate (PEGDA), polyethylene glycol dimethacrylate (PEGDMA), and combinations thereof.

6. The hydrogel sensor device of claim 2, wherein the crosslinking monomer is present in the crosslinked hydrogel network in an amount of from about 0.01 mol % to about 10 mol %.

7. The hydrogel sensor device of claim 1, wherein the first affinity ligand and the second affinity ligand are different.

8. The hydrogel sensor device of claim 1, wherein the first affinity ligand is configured to bind to a first portion of the target molecule and the second affinity ligand is configured to bind to a second portion of the target molecule.

9. The hydrogel sensor device of claim 8, wherein the first portion is the crystallizable fragment (Fc) region of an antibody.

10. The hydrogel sensor device of claim 9, wherein the first affinity ligand is protein A, protein G, protein AG, protein Z, protein LG, protein LA, peptide PAM, peptide FcBP-1, peptide FcBP-2, peptide FcRM, peptide Fc-III, or peptide Fc-III-4C.

11. The hydrogel sensor device of claim 8, wherein the second portion of the target molecule is the antigen-binding fragment (Fab) region of an antibody.

12. The hydrogel sensor device of claim 11, wherein the second affinity ligand is protein A, protein G, protein L, or ligand 8/7.

13. The hydrogel sensor device of claim 1, wherein the first affinity ligand is present in the crosslinked hydrogel network in an amount of from about 0.001 mol % to about 1 mol %.

14. The hydrogel sensor device of claim 1, wherein the second affinity ligand is present in the crosslinked hydrogel network in an amount of from about 0.001 mol % to about 1 mol %.

15. The hydrogel sensor device of claim 11, wherein the target molecule is selected from the group consisting of a virus, a protein, a nucleic acid, an enzyme, an antibody, and combinations thereof

16. The hydrogel sensor device of claim 1, wherein the detector is selected from an optical detector, a pressure detector, a magnetometer, and combinations thereof

17. The hydrogel sensor device of claim 1, wherein the change in volume is detected by collection of a detector response signal and correlation of the detector response signal with a reference dataset.

18. A method of detecting a target molecule, comprising:
concurrently and reversibly binding a common target molecule to a first affinity ligand and a second affinity ligand of a crosslinked hydrogel network to decrease a volume of the crosslinked hydrogel network, wherein one or both of the first affinity ligand and the second affinity ligand are at least one of a protein, a protein fragment, a peptide, a synthetic biomimetic ligand, and or a synthetic chemical ligand; and
detecting changes in the volume.

19. The method of claim 18, wherein detecting is performed with an optical detector, a pressure detector, a magnetometer, or a combination thereof

20. The method of claim 18, further comprising correlating changes in volume with a local concentration of the common target molecule.

21. The method of claim 20, further comprising transmitting local concentration data to a remote device.
